(19) United States
(12) Patent Application Publication
(10) Pub. No.: US 2026/0345109 A1
(43) Pub. Date: Nov. 5, 2026

(54) EXPOSURE PARAMETER DETERMINATION

(71) Applicant: Siemens Healthineers AG, Forchheim (DE)

(72) Inventors: Chen Jie Ge, Shanghai (CN); Jing Tai Cao, Shanghai (CN); Xi Shuai Peng, Shanghai (CN); Yun Zhe Zhe Zou, Shanghai (CN)

(73) Assignee: Siemens Healthineers AG, Forchheim (DE)

(21) Appl. No.: 19/101,760

(22) PCT Filed: Aug. 9, 2023
(86) PCT No.: PCT/EP2023/072074

§ 371 Date: Feb. 6, 2025

(30) Foreign Application Priority Data
Aug. 9, 2022 (CN) 202210953947.9

(51) Int. Cl. A61B 6/00; G06T 7/50

(52) U.S. Cl. CPC A61B 6/544; G06T 7/50; G06T 2200/04; G06T 2207/20081; G06T 2207/20084; G06T 2207/30004

(57) ABSTRACT

A method and apparatus for determining exposure parameters in X-ray imaging, a storage medium, and a program product. The method includes: acquiring a 3D image of a subject; based on the 3D image, extracting a body shape feature of the subject; inputting the body shape feature into an exposure parameter prediction model corresponding to an X-ray imaging protocol, wherein the exposure parameter prediction model is obtained by training based on first training data, the first training data including a historical body shape feature in a historical exposure operation of the X-ray imaging protocol and an exposure parameter historical value in the historical exposure operation; receiving, from the exposure parameter prediction model, an exposure parameter predicted value determined on the basis of the body shape feature.

### TECHNICAL FIELD

The present disclosure relates to the technical field of medical imaging, in particular to a method and apparatus for determining exposure parameters in X-ray imaging, a storage medium, and a program product.

### BACKGROUND ART

X-rays are electromagnetic radiation with a wavelength between ultraviolet rays and gamma rays. X-rays are penetrating, having different penetrating abilities for substances of different densities. In medical settings, X-rays are generally used to project organs and bones of the human body to form medical images. An X-ray imaging system generally comprises an X-ray generating component, a Bucky Wall Stand (BWS) component, an examination table component, a cassette component comprising a plate detector, and a control master computer located remotely, etc. The X-ray generating component uses a high voltage provided by a high voltage generator to emit X-rays, which pass through an irradiated imaging target and form medical image information of the imaging target on the plate detector. The plate detector sends the medical image information to the control master computer. The imaging target can stand close to the Bucky wall stand component or lie on the examination table component so as to separately undergo X-ray photography of parts such as the head, chest, abdomen, and joints.

In X-ray imaging, X-ray exposure parameters (such as tube voltage, tube current, and exposure time, etc.) have a major impact on X-ray image quality. At present, X-ray exposure parameters are mainly set independently according to the technician's personal experience, but this has the drawback of difficulty of implementation.

### SUMMARY

Aspects of the present disclosure introduce a method and apparatus for determining exposure parameters in X-ray imaging, as well as a storage medium and a program product.

A method for determining exposure parameters in X-ray imaging, the method comprising:
acquiring a 3D image of a subject;
based on the 3D image, extracting a body shape feature of the subject;
inputting the body shape feature into an exposure parameter prediction model corresponding to an X-ray imaging protocol, wherein the exposure parameter prediction model is obtained by training based on first training data, the first training data including a historical body shape feature in a historical exposure operation of the X-ray imaging protocol and an exposure parameter historical value in the historical exposure operation;
receiving, from the exposure parameter prediction model, an exposure parameter predicted value determined on the basis of the body shape feature.

Thus, aspects of the present disclosure automatically determine exposure parameters according to the 3D image of the subject, increasing the accuracy of exposure and reducing manual difficulty. Moreover, in the process of automatically determining exposure parameters, the association between body shape features of the subject and exposure parameters is also taken into account, further increasing the accuracy of parameters.

In a demonstrative aspect, the method further comprises:
establishing a first artificial neural network model;
using the first training data to train the first artificial neural network model into the exposure parameter prediction model, wherein the training comprises:

inputting the first training data into the first artificial neural network model, so that the first artificial neural network model outputs an exposure parameter predicted value corresponding to the first training data; based on the difference between the exposure parameter historical value in the first training data and the exposure parameter predicted value corresponding to the first training data, determining a loss function value of the first artificial neural network model;
configuring a model parameter of the first artificial neural network model, so that the loss function value is lower than a first preset threshold; and
determining the first artificial neural network model resulting from the configuration to be the exposure parameter prediction model.

Thus, by introducing an artificial neural network into the process of exposure parameter prediction, the difficulty of implementation is reduced.

In a demonstrative aspect, the step of extracting a body shape feature of the subject based on the 3D image comprises:
inputting the 3D image into a body shape feature determining model, wherein the body shape feature determining model is obtained by training based on second training data, the second training data including a historical 3D image and a body shape feature labeled on the basis of the historical 3D image;
receiving, from the body shape feature determining model, a body shape feature determined on the basis of the 3D image.

Thus, automatic extraction of body shape features from the 3D image is achieved, increasing the efficiency of feature extraction.

In a demonstrative aspect, the method further comprises:
establishing a second artificial neural network model;
using the second training data to train the second artificial neural network model into the body shape feature determining model, the training comprising:
inputting the second training data into the second artificial neural network model so that the second artificial neural network model outputs a predicted body shape feature corresponding to the second training data; based on the difference between the labeled body shape feature in the second training data and the predicted body shape feature, determining a loss function value of the second artificial neural network model; configuring a model parameter of the second artificial neural network model, so that the loss function value is lower than a second preset threshold; and determining the second artificial neural network model resulting from the configuration to be the body shape feature determining model.

Thus, by introducing an artificial neural network into the process of body shape feature extraction, the difficulty of implementation is reduced.

In a demonstrative aspect, the method further comprises:
judging whether an adjustment instruction for the exposure parameter predicted value is received, wherein, when an adjustment instruction for the exposure parameter predicted value is received, the exposure parameter predicted value is adjusted on the basis of the adjustment instruction, and the imaging target is subjected to a first exposure operation on the basis of the adjusted exposure parameter predicted value; when no adjustment instruction for the exposure parameter predicted value is received, the imaging target is subjected to a second exposure operation on the basis of the exposure parameter predicted value.

Thus, the exposure parameter predicted value can be adjusted on the basis of an adjustment operation, ensuring the accuracy of the predicted value.

In a demonstrative aspect, the method further comprises:
after executing the first exposure operation, storing third training data in a log, the third training data including the adjusted exposure parameter predicted value for executing the first exposure operation and a body shape feature of the imaging target;
extracting the third training data from the log;
using the third training data to subject the exposure parameter prediction model to an update training process, the update training process comprising:
inputting the third training data into the exposure parameter prediction model, so that the exposure parameter prediction model outputs an exposure parameter predicted value corresponding to the third training data;
based on the difference between the adjusted exposure parameter predicted value in the third training data and the exposure parameter predicted value corresponding to the third training data, determining a loss function value of the exposure parameter prediction model;
configuring a model parameter of the exposure parameter prediction model, so that the loss function value is lower than a third preset threshold; and
determining the exposure parameter prediction model resulting from the configuration to be an exposure parameter prediction model resulting from update training.

Clearly, as the exposure parameter prediction model is subjected to update training by means of the log, adjustment preferences can be introduced into the exposure parameter prediction model, so that the exposure parameter prediction model conforms more closely to user habits.

In a demonstrative aspect, the body shape feature comprises a first body shape feature having a first weighting and a second body shape feature having a second weighting, wherein the first body shape feature characterizes an overall body shape of the subject, and the second body shape feature characterizes a local body shape of an imaging target, which is included in the subject and corresponds to the X-ray imaging protocol, wherein the second weighting is greater than the first weighting.

Thus, the body shape feature of the subject and the body shape feature of the imaging target are used together to obtain a prediction result. The prediction result takes full account of the overall body shape and the local body shape, ensuring accuracy. Moreover, the weighting of the local body shape is greater than the weighting of the overall body shape, improving the association between the prediction result and the imaging target.

An apparatus for determining exposure parameters in X-ray imaging, the apparatus comprising:
an acquisition module, configured to acquire a 3D image of an imaging target;
an extraction module, configured to extract a body shape feature of the imaging target on the basis of the 3D image;
an input module, configured to input the body shape feature into an exposure parameter prediction model corresponding to an X-ray imaging protocol, wherein the exposure parameter prediction model is obtained by training based on first training data, the first training data including a historical body shape feature in a historical exposure operation of the X-ray imaging protocol and an exposure parameter historical value in the historical exposure operation;
a receiving module, configured to receive, from the exposure parameter prediction model, an exposure parameter predicted value determined on the basis of the body shape feature.

The apparatus is, in particular, implemented to carry out the method/steps as disclosed above or below with respect to aspects of the method and/or the method according to the appended claims.

Thus, aspects of the present disclosure automatically determine exposure parameters according to the 3D image of the subject, increasing the accuracy of exposure and reducing manual difficulty. Moreover, in the process of automatically determining exposure parameters, the association between body shape features of the subject and exposure parameters is also taken into account, further increasing the accuracy of parameters.

In a demonstrative aspect, the extraction module is configured to input the 3D image into a body shape feature determining model, wherein the body shape feature determining model is obtained by training based on second training data, the second training data including a historical 3D image and a body shape feature labeled on the basis of the historical 3D image; and receive, from the body shape feature determining model, a body shape feature determined on the basis of the 3D image.

In a demonstrative aspect, the apparatus further comprises:
an exposure module configured to judge whether an adjustment instruction for the exposure parameter predicted value is received, wherein, when an adjustment instruction for the exposure parameter predicted value is received, the exposure parameter predicted value is adjusted on the basis of the adjustment instruction, and the imaging target is subjected to a first exposure operation on the basis of the adjusted exposure parameter predicted value; when no adjustment instruction for the exposure parameter predicted value is received, the imaging target is subjected to a second exposure operation on the basis of the exposure parameter predicted value.

Thus, the exposure parameter predicted value can be adjusted on the basis of an adjustment operation, ensuring the accuracy of the predicted value.

In a demonstrative aspect, the apparatus further comprises:
an update module, configured to store third training data in a log after the first exposure operation is executed, the third training data including the adjusted exposure parameter predicted value for executing the first exposure operation and a body shape feature of the imaging target;
extracting the third training data from the log; using the third training data to subject the exposure parameter prediction model to an update training process, the update training process comprising: inputting the third training data into the exposure parameter prediction model so that the exposure parameter prediction model outputs an exposure parameter predicted value corresponding to the third training data; based on the difference between the adjusted exposure parameter predicted value in the third training data and the exposure parameter predicted value corresponding to the third training data, determining a loss function value of the exposure parameter prediction model;
configuring a model parameter of the exposure parameter prediction model so that the loss function value is lower than a third preset threshold; and determining the exposure parameter prediction model resulting from the configuration to be an exposure parameter prediction model resulting from update training.

Clearly, as the exposure parameter prediction model is subjected to update training by means of the log, adjustment preferences can be introduced into the exposure parameter prediction model so that the exposure parameter prediction model conforms more closely to user habits.

An apparatus for determining exposure parameters in X-ray imaging, comprising a processor and a memory;
an application program executable by the processor is stored in the memory and used to cause the processor to perform the method for determining exposure parameters in X-ray imaging as described in any one of the aspects above.

A computer-readable storage medium, having stored therein a computer-readable instruction which, when executed by a processor, realizes the method for determining exposure parameters in X-ray imaging as described in any one of the aspects above.

A computer program product, comprising a computer program which, when executed by a processor, realizes the method for determining exposure parameters in X-ray imaging as described in any one of the aspects above.

### BRIEF DESCRIPTION OF THE DRAWINGS

Preferred aspects of the present disclosure are described in detail below with reference to the drawings to give those skilled in the art a clearer understanding of the abovementioned and other features and advantages of the present disclosure. In the figures:

FIG. 1 is a demonstrative flow chart of a method for determining exposure parameters in X-ray imaging according to aspects of the present disclosure.

FIG. 2 is a demonstrative flow chart of determining body shape features of a subject according to aspects of the present disclosure.

FIG. 3 is a schematic diagram of exposure operation log storage according to aspects of the present disclosure.

FIG. 4 is a schematic diagram of a demonstrative process of determining exposure parameters in X-ray imaging according to aspects of the present disclosure.

FIG. 5 is a demonstrative structural diagram of an apparatus for determining exposure parameters in X-ray imaging according to aspects of the present disclosure.

FIG. 6 is a demonstrative structural diagram of an apparatus for determining exposure parameters in X-ray imaging according to aspects of the present disclosure.

### DETAILED DESCRIPTION

To clarify the objective, technical solution and advantages of the present disclosure, the present disclosure is explained in further detail below by way of aspects.

The solution of the present disclosure is expounded below by describing a number of representative aspects, in order to make the description concise and intuitive. The large number of details in the aspects are merely intended to assist with understanding the solution of the present disclosure. However, obviously, the technical solution of the present disclosure need not be limited to these details when implemented. To avoid making the solution of the present disclosure unnecessarily confusing, some aspects are not described meticulously but merely outlined. Hereinbelow, “comprises” means “including but not limited to,” while “according to . . . ” means “at least according to . . . , but not limited to only according to . . . ”. In line with the linguistic customs of Chinese, in cases where the quantity of a component is not specified hereinbelow, this means that there may be one or more of the component; this may also be interpreted as meaning at least one.

In view of the drawback in the prior art that X-ray exposure parameters are set independently according to the technician's personal experience, aspects of the present disclosure take into account the association between body shape features of a subject and exposure parameters (in general, the larger the body shape of the subject, the greater the exposure parameters), automatically determine body shape features according to a 3D image of the subject, and then automatically determine exposure parameters based on the body shape features, thus increasing the exposure accuracy and reducing manual difficulty.

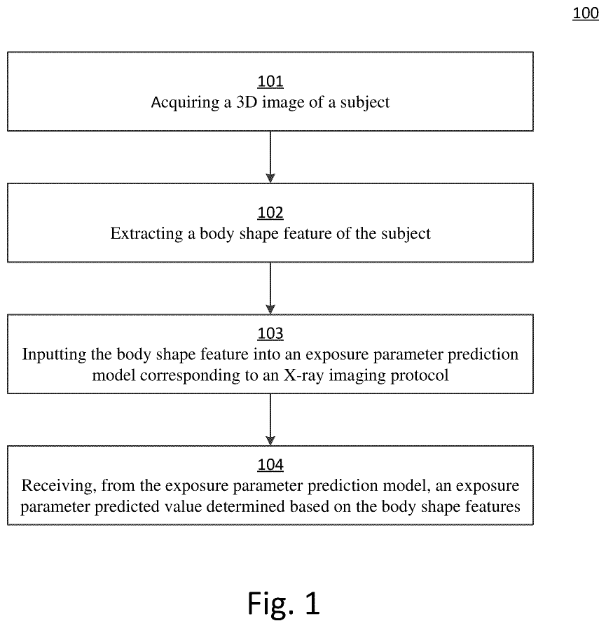

FIG. 1 is a flow chart of a method for determining exposure parameters in X-ray imaging according to aspects of the present disclosure. Preferably, the method shown in FIG. 1 may be performed by a controller. The controller may be implemented as a control master computer integrated into an X-ray imaging system or may be implemented as a control unit that is independent of a control master computer.

As shown in FIG. 1, the method 100 comprises:

Step 101: acquiring a 3D image of a subject.

In an aspect, a camera component may be used in step 101 to photograph the subject so as to obtain a 3D image of the subject. In another aspect, a 3D image of the subject may be acquired from a storage medium (e.g., the Cloud or a local database) in step 101, wherein the 3D image is obtained by using a camera component to photograph the subject.

Here, a light source of the camera component may or may not coincide with an X-ray source in the X-ray imaging system. When the light source of the camera component coincides with the X-ray source in the X-ray imaging system, the camera component is generally fixed to a beam limiter housing or tube cover of the X-ray generating component. For example, a recess for accommodating the camera component is arranged on the tube cover or on the housing of the beam limiter, and the camera component is fixed to the recess by bolt connection, a snap-fit connection, a steel wire loop, etc. When the light source of the camera component does not coincide with the X-ray source in the X-ray imaging system, the camera component may be arranged at any position suitable for photographing the subject, in an examination room in which the subject is located, e.g., on the ceiling, on the floor, or on various components in the medical imaging system, etc.

In one aspect, the camera component comprises at least one 3D camera. The 3D camera uses 3D imaging technology to photograph the subject, so as to generate a 3D image of the subject.

In one aspect, the camera component comprises at least two 2D cameras, each of which is separately arranged at a predetermined position. In practice, those skilled in the art can select a suitable position as the predetermined position to arrange the 2D camera as required. The camera component may further comprise an image processor. The image processor synthesizes a 3D image of the subject from 2D images captured by the 2D cameras, wherein a depth of field used by the image processor during synthesis may be a depth of field of any of the 2D images. Optionally, each 2D camera may send its respectively captured 2D image to an image processor outside the camera component, for the image processor outside the camera component to synthesize a 3D image of the subject from the 2D images captured by the 2D cameras, wherein a depth of field used by the image processor outside the camera component during synthesis may likewise be a depth of field of any of the 2D images. Specifically, the image processor outside the camera component may be implemented as a control master computer in the X-ray imaging system, or as an independent control unit separate from the X-ray imaging system. Each 2D camera may be arranged at any position suitable for photographing the subject, in an examination room in which the subject is located, e.g., on the ceiling, on the floor, or on various components in the X-ray imaging system, etc.

In one aspect, the camera component may comprise: at least one 2D camera and at least one depth of field sensor. The at least one 2D camera and at least one depth of field sensor are installed at the same position. The camera component may further comprise an image processor. The image processor uses a depth of field provided by the depth of field sensor and a 2D photograph provided by the 2D camera together to generate a 3D image of the subject. Optionally, the 2D camera sends a captured 2D image of the subject to an image processor outside the camera component, and the depth of field sensor sends an acquired depth of field to the image processor outside the camera component for the image processor outside the camera component to use the depth of field and the 2D photograph together to generate a 3D image of the subject. Preferably, the image processor outside the camera component may be implemented as a control master computer in the X-ray imaging system or as an independent control unit separate from the X-ray imaging system. The 2D camera may be arranged at any position suitable for photographing the subject in an examination room in which the subject is located, e.g., on the ceiling, on the floor, or on various components in the X-ray imaging system, etc.

After acquiring the 3D image of the subject, the camera component may send the 3D image via a wired interface or wireless interface to a controller which performs the procedure in FIG. 1. Preferably, the wired interface comprises at least one of the following: a universal serial bus interface, controller local area network interface or serial port, etc.; the wireless interface comprises at least one of the following: an infrared interface, near field communication interface, Bluetooth interface, Zigbee interface, wireless broadband interface, etc.

Typical examples of the camera component photographing the subject to generate a 3D image have been described demonstratively above, but those skilled in the art will realize that such descriptions are merely demonstrative and not intended to define the scope of protection of the aspects of the present disclosure.

Step 102: based on the 3D image, extracting body shape features of the subject.

Body shape is an overall description and assessment of the shape of the human body, and mainly includes the length and width of each part as well as the proportions of parts relative to one another. There is a certain relationship between body shape and the subject's movement ability and other functions. In general, the larger the subject's body shape, the greater the exposure parameters.

The body shape features are used to characterize the subject's body shape. In an aspect, the body shape features comprise a first body shape feature characterizing the overall body shape of the subject. For example, the first body shape feature may comprise: body height, body weight, upper limb length, lower limb length, upper/lower limb length ratio, chest circumference, waist circumference, hip circumference, waist/hip ratio, height waist index and body mass index (BMI), etc.

In an aspect, the body features comprise a second body shape feature, which characterizes the body shape of an imaging target included in the subject and corresponds to an X-ray imaging protocol. The imaging target is a target in the subject and needs to be subjected to X-ray imaging based on the X-ray imaging protocol. For example, the imaging target may be a palm of a hand, the waist, the abdomen, or the spine of the subject, etc. The X-ray imaging protocol is a specific protocol (e.g., tissue organ protocol (OGP)) used in the process of subjecting the imaging target to X-ray imaging. The X-ray imaging protocol may be determined on the basis of a selection operation of a user in a human-machine interface. For example, when the imaging target is the subject's abdomen, the second body shape feature may comprise: abdomen fat thickness, infrasternal angle, position of the upper boundary of the abdomen, etc. When the imaging target is the subject's spine, the second body shape feature may comprise: spine length, vertebral body length, intervertebral disk length, and ratio of spine length to body height, etc.

Typical examples of the first body shape feature and the second body shape feature have been described demonstratively above, but those skilled in the art will realize that such descriptions are merely demonstrative and not intended to define the scope of protection of the aspects of the present disclosure.

In an aspect, the step of extracting body shape features of the subject based on the 3D image comprises: inputting the 3D image into a body shape feature determining model, wherein the body shape feature determining model is obtained by training based on second training data, the second training data including historical 3D images and body shape features labelled on the basis of the historical 3D images; and receiving, from the body shape feature determining model, body shape features determined on the basis of the 3D image. Thus, automatic extraction of body shape features from the 3D image is achieved, increasing the efficiency of feature extraction. The historical 3D images may comprise: 3D images of subjects in exposure operations that have already been executed historically using the X-ray imaging protocol; the body shape features labelled on the basis of the historical 3D images may be: body shape features labelled after manually browsing the historical 3D images, or body shape features labelled automatically on the basis of a machine algorithm, etc. In an aspect, the method further comprises: establishing a second artificial neural network model; and using second training data to train the second artificial neural network model into a body shape feature determining model. The training comprises: inputting second training data into the second artificial neural network model, so that the second artificial neural network model outputs a predicted body shape feature corresponding to the second training data; based on the difference between a labelled body shape feature in the second training data and the predicted body shape feature, determining a loss function value of the second artificial neural network model; configuring model parameters of the second artificial neural network model, so that the loss function value is lower than a second preset threshold; and determining the second artificial neural network model resulting from the configuration to be the body shape feature determining model. Thus, by introducing an artificial neural network into the process of body shape feature extraction, the difficulty of implementation is reduced.

In another aspect, a 3D model of the subject is obtained by simulation modeling, using the 3D image of the subject as modeling data. Next, body shape features of the subject are read from the 3D model. For example, simulation modeling comprises: wire frame modeling, solid modeling, surface modeling, etc. For example, in surface modeling, matching mark points or image contours are extracted from the 3D image, and human body 3D deformation and motion parameters are estimated according to the mark points and image contours as well as the constraint that the volume does not change, then spheres and conic surfaces of revolution are used to draw a human body model.

Step 103: inputting the body shape features into an exposure parameter prediction model corresponding to an X-ray imaging protocol, wherein the exposure parameter prediction model is obtained by training based on first training data, the first training data including historical body shape features in historical exposure operations of the X-ray imaging protocol and exposure parameter historical values in the historical exposure operations.

In an aspect, the method further comprises: establishing a first artificial neural network model; using first training data to train the first artificial neural network model into an exposure parameter prediction model, wherein the training comprises: inputting the first training data into the first artificial neural network model, so that the first artificial neural network model outputs an exposure parameter predicted value corresponding to the first training data; based on the difference between an exposure parameter historical value in the first training data and the exposure parameter predicted value corresponding to the first training data, determining a loss function value of the first artificial neural network model; configuring model parameters of the first artificial neural network model, so that the loss function value is lower than a first preset threshold; and determining the first artificial neural network model resulting from the configuration to be the exposure parameter prediction model. In the process of training, the first artificial neural network model automatically learns the association between historical body shape features and exposure parameter historical values and has the ability to use body shape features to predict exposure parameters. The meaning of historical exposure operations of the X-ray imaging protocol is: exposure operations that have already been executed historically using the X-ray imaging protocol. Body shape features of subjects in historical exposure operations (referred to as historical body shape features) and exposure parameters in historical exposure operations (referred to as exposure parameter historical values) are acquired. The first training data is constructed on the basis of historical body shape features and exposure parameter historical values.

Specifically, the exposure parameters may comprise: at least one of tube voltage, tube current, exposure time, exposure dose, the product of tube current and exposure time, and exposure density.

Thus, the association between body shape features and exposure parameters is contained in the exposure parameter prediction model, which is trained using exposure parameter historical values in historical exposure operations and historical body shape features in historical exposure operations. Therefore, the exposure parameter prediction model can increase the accuracy of exposure parameters.

Step 104: receiving, from the exposure parameter prediction model, exposure parameter predicted values determined on the basis of the body shape features.

Thus, aspects of the present disclosure automatically determine exposure parameters according to the 3D image of the subject, increasing the accuracy of exposure and reducing manual difficulty. Moreover, in the process of automatically determining exposure parameters, the association between body shape features of the subject and exposure parameters is also taken into account, further increasing the accuracy of parameters.

In an aspect, the body shape features comprise a first body shape feature having a first weighting and a second body shape feature having a second weighting, wherein the second weighting is greater than the first weighting. Correspondingly, historical body shape features in first training data, second training data and third training data also correspondingly include a first historical body shape feature having a first weighting and a second historical body shape feature having a second weighting. Clearly, overall body shape features of the subject and local body shape features of the imaging target are used together to obtain a prediction result. The prediction result takes full account of the overall body shape of the subject and the local body shape of the imaging target, ensuring accuracy. Moreover, the weighting of the local body shape is greater than the weighting of the overall body shape, improving the association between the prediction result and the imaging target.

In an aspect, the method further comprises: displaying an exposure parameter predicted value (e.g., via a user interface); judging whether an adjustment instruction for the exposure parameter predicted value is received (e.g., via the user interface), wherein, when an adjustment instruction for the exposure parameter predicted value is received, the exposure parameter predicted value is adjusted on the basis of the adjustment instruction, and the imaging target is subjected to a first exposure operation on the basis of the adjusted exposure parameter predicted value; when no adjustment instruction for the exposure parameter predicted value is received, the imaging target is subjected to a second exposure operation on the basis of the exposure parameter predicted value. Thus, the exposure parameter predicted value can be adjusted on the basis of an adjustment operation, ensuring the accuracy of the predicted value.

In an aspect, the method further comprises: storing third training data in a log, the third training data including exposure parameter values for executing an exposure operation and body shape features of an imaging target; extracting the third training data from the log; using the third training data to subject the exposure parameter prediction model to an update training process, the update training process comprising: inputting the third training data into the exposure parameter prediction model, so that the exposure parameter prediction model outputs an exposure parameter predicted value corresponding to the third training data; based on the difference between an exposure parameter value in the third training data and the exposure parameter predicted value corresponding to the third training data, determining a loss function value of the exposure parameter prediction model; configuring model parameters of the exposure parameter prediction model, so that the loss function value is lower than a third preset threshold; and determining the exposure parameter prediction model resulting from the configuration to be an exposure parameter prediction model resulting from update training. Clearly, as the exposure parameter prediction model is subjected to update training by means of the log, adjustment preferences can be introduced into the exposure parameter prediction model, so that the exposure parameter prediction model conforms more closely to user habits.

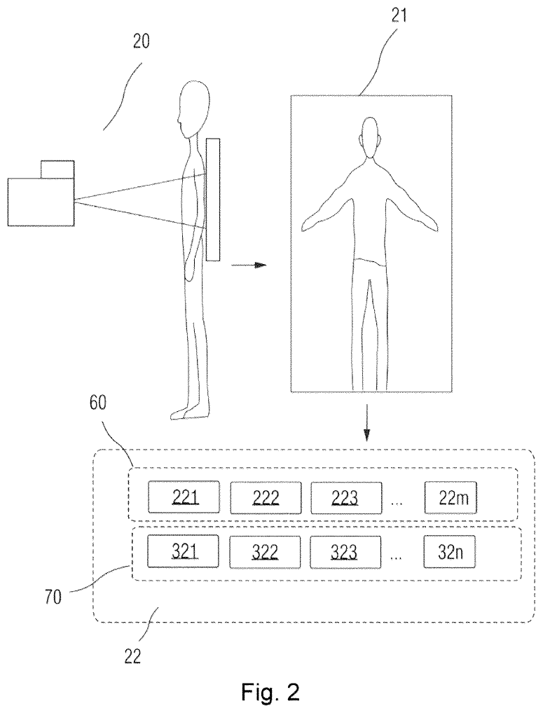

FIG. 2 is a demonstrative flow chart of determining body shape features of a subject according to aspects of the present disclosure.

First of all, photography processing 20 is performed. In the photography processing 20, a 3D camera is used to photograph the subject, so as to obtain a 3D image of the subject. A 3D model 21 of the subject is then obtained by simulation modeling, using the 3D image of the subject as modeling data. Next, body shape features 22 of the subject are extracted from the 3D model 21. The body shape features 22 comprise: (1) overall body shape features 60 of the subject; (2) body shape features 70 of an imaging target which is included in the subject and corresponds to an X-ray imaging protocol—these may be called local body shape features. There may be one or more overall body shape features 60; for example, they specifically comprise the subject's upper/lower limb length ratio 221, the chest circumference 222, and the waist circumference 223, etc. There may be one or more local body shape features 70. For example, when the imaging target is the abdomen, the local body shape features 70 may comprise the abdomen fat thickness 321, infrasternal angle 322, position of the upper boundary of the abdomen 323, etc.

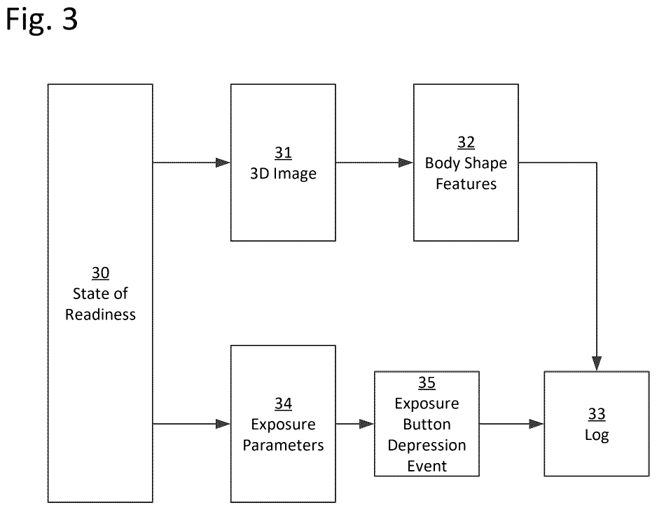

FIG. 3 is a schematic diagram of log storage according to aspects of the present disclosure. When the subject has entered a state of readiness 30, the subject is photographed to obtain a 3D image 31. The 3D image 31 is used to extract body shape features 32. Exposure parameters 34 are set on a setting interface of a control master computer. After detecting an exposure button depression event 35, the exposure parameters 34 and the body shape features 32 are stored in a log 33 in an associated manner. Subsequently, the exposure parameters 34 and the body shape features 32 can be extracted from the log 33, to serve as first training data of an exposure parameter prediction model.

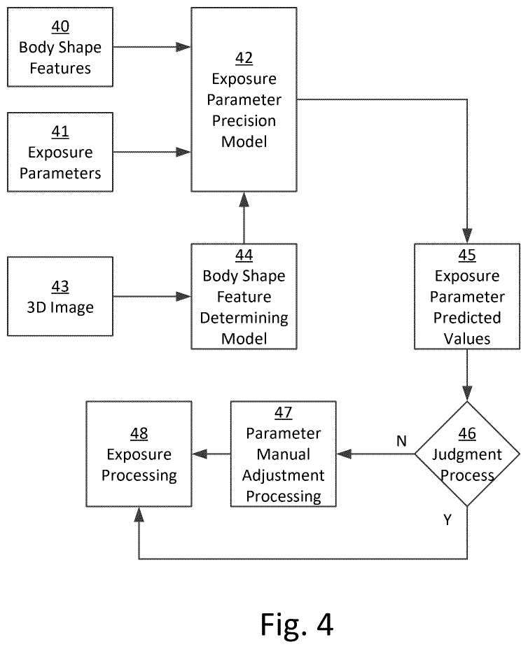

FIG. 4 is a schematic diagram of a demonstrative process of determining exposure parameters in X-ray imaging according to aspects of the present disclosure.

An exposure parameter prediction model 42 is obtained by training in advance. Specifically, this comprises: using body shape features 40 saved in a log and exposure parameters 41 associated with the body shape features 40 as first training data. Here, an association between body shape features 40 and exposure parameters 41 means that: the exposure parameters 41 have already been used to subject a historical subject having the body shape features 40 to exposure. The first training data is inputted into a first artificial neural network model, so that the first artificial neural network model outputs exposure parameter predicted values corresponding to the first training data; based on the difference between exposure parameter historical values in the first training data and the exposure parameter predicted values corresponding to the first training data, a loss function value of the first artificial neural network model is determined; model parameters of the first artificial neural network model are configured, so that the loss function value is lower than a first preset threshold; and the first artificial neural network model resulting from the configuration is determined to be the exposure parameter prediction model 42.

After obtaining the exposure parameter prediction model 42 by training, the exposure parameter prediction model 42 may be used to automatically determine exposure parameters. The specific process of determining exposure parameters comprises: using a camera component to photograph a subject, to obtain a 3D image 43 of the subject. The 3D image 43 is inputted into a body shape feature determining model 44, wherein the body shape feature determining model 44 is obtained by training based on second training data, the second training data including historical 3D images and body shape features labelled on the basis of the historical 3D images. The body shape feature determining model 44 determines body shape features of the subject on the basis of the 3D image of the subject. The body shape feature determining model 44 sends the body shape features of the subject to the exposure parameter prediction model 42. The exposure parameter prediction model 42 obtains exposure parameter predicted values 45 by prediction based on the body shape features of the subject. The exposure parameter predicted values 45 are displayed on a user interface. Then in a judgment process 46, a judgment is made as to whether a confirmation instruction for the exposure parameter predicted values 45 displayed on the user interface is received, and if so (corresponding to the branch “Y”), exposure processing 48 is performed; otherwise (corresponding to the branch “N”), exposure 48 parameter manual adjustment processing 47 is performed. In the exposure parameter manual adjustment processing 48, the exposure parameter predicted values 45 are manually adjusted, then the manually adjusted exposure parameter predicted values 45 are used to perform exposure processing 48.

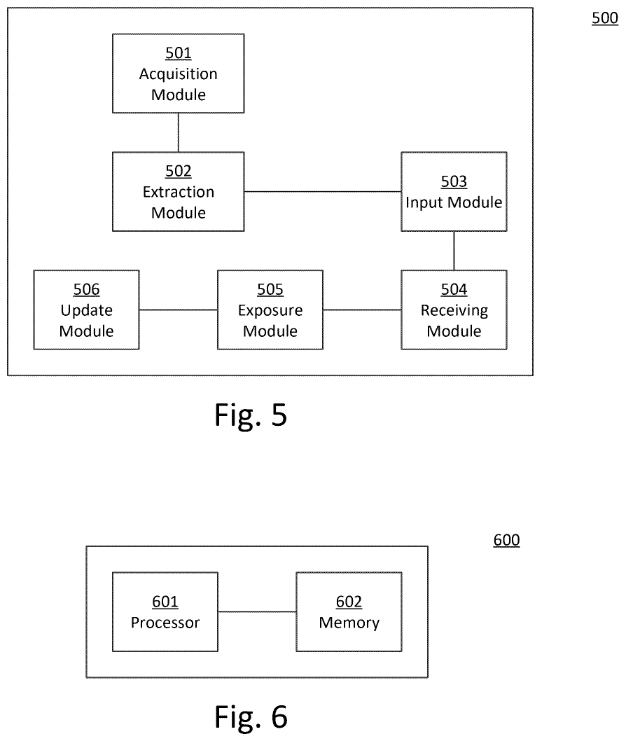

FIG. 5 is a structural diagram of an apparatus for determining exposure parameters in X-ray imaging according to aspects of the present disclosure.

As shown in FIG. 5, an apparatus 500 for determining exposure parameters in X-ray imaging comprises: an acquisition module 501, configured to acquire a 3D image of an imaging target; an extraction module 502, configured to extract body shape features of the imaging target on the basis of the 3D image; an input module 503, configured to input the body shape features into an exposure parameter prediction model corresponding to an X-ray imaging protocol, wherein the exposure parameter prediction model is obtained by training based on first training data, the first training data including historical body shape features in historical exposure operations of the X-ray imaging protocol and exposure parameter historical values in the historical exposure operations; and a receiving module 504, configured to receive, from the exposure parameter prediction model, exposure parameter predicted values determined on the basis of the body shape features.

In a demonstrative aspect, the apparatus 500 further comprises: a training module 506, configured to establish a first artificial neural network model; and use first training data to train the first artificial neural network model into an exposure parameter prediction model, wherein the training comprises: inputting the first training data into the first artificial neural network model, so that the first artificial neural network model outputs an exposure parameter predicted value corresponding to the first training data; based on the difference between an exposure parameter historical value in the first training data and the exposure parameter predicted value corresponding to the first training data, determining a loss function value of the first artificial neural network model; configuring model parameters of the first artificial neural network model, so that the loss function value is lower than a first preset threshold; and determining the first artificial neural network model resulting from the configuration to be the exposure parameter prediction model.

In a demonstrative aspect, the extraction module 502 is configured to input the 3D image into a body shape feature determining model, wherein the body shape feature determining model is obtained by training based on second training data, the second training data including historical 3D images and body shape features labelled on the basis of the historical 3D images; and receive, from the body shape feature determining model, body shape features determined on the basis of the 3D image.

In a demonstrative aspect, the training module 506 is configured to establish a second artificial neural network model; and use second training data to train the second artificial neural network model into a body shape feature determining model, the training comprising: inputting the second training data into the second artificial neural network model, so that the second artificial neural network model outputs a predicted body shape feature corresponding to the second training data; based on the difference between a labelled body shape feature in the second training data and the predicted body shape feature, determining a loss function value of the second artificial neural network model; configuring model parameters of the second artificial neural network model, so that the loss function value is lower than a second preset threshold; and determining the second artificial neural network model resulting from the configuration to be the body shape feature determining model.

In a demonstrative aspect, the apparatus further comprises an exposure module 507, configured to judge whether an adjustment instruction for the exposure parameter predicted value is received, wherein, when an adjustment instruction for the exposure parameter predicted value is received, the exposure parameter predicted value is adjusted on the basis of the adjustment instruction, and the imaging target is subjected to an exposure operation on the basis of the adjusted exposure parameter predicted value; when no adjustment instruction for the exposure parameter predicted value is received, the imaging target is subjected to an exposure operation on the basis of the exposure parameter predicted value.

In a demonstrative aspect, the apparatus further comprises: an update module 508, configured to store third training data in a log, the third training data including exposure parameter values for executing an exposure operation and body shape features of an imaging target; extract the third training data from the log; and use the third training data to subject the exposure parameter prediction model to an update training process, the update training process comprising: inputting the third training data into the exposure parameter prediction model, so that the exposure parameter prediction model outputs an exposure parameter predicted value corresponding to the third training data; based on the difference between an exposure parameter value in the third training data and the exposure parameter predicted value corresponding to the third training data, determining a loss function value of the exposure parameter prediction model; configuring model parameters of the exposure parameter prediction model, so that the loss function value is lower than a third preset threshold; and determining the exposure parameter prediction model resulting from the configuration to be an exposure parameter prediction model resulting from update training.

In a demonstrative aspect, the body shape features comprise a first body shape feature having a first weighting and a second body shape feature having a second weighting, wherein the first body shape feature characterizes the overall body shape of the subject, and the second body shape feature characterizes a local body shape of an imaging target, which is included in the subject and corresponds to an X-ray imaging protocol.

FIG. 6 is a structural diagram of an apparatus having a memory-processor architecture for determining exposure parameters in X-ray imaging according to aspects of the present disclosure.

As shown in FIG. 6, the apparatus 600 for determining exposure parameters in X-ray imaging comprises a processor 601, a memory 602, and a computer program that is stored on the memory 602 and capable of being run on the processor 601; when executed by the processor 601, the computer program implements the method for determining exposure parameters in X-ray imaging according to any one of the aspects above. The memory 602 may specifically be implemented as various types of storage media, such as an electrically erasable programmable read-only memory (EEPROM), a flash memory or a programmable read-only memory (PROM). The processor 601 may be implemented as comprising one or more central processors or one or more field-programmable gate arrays, wherein the field programmable gate array integrates one or more central processor cores. Specifically, the central processor or central processor core may be implemented as a CPU or MCU or DSP, etc.

It must be explained that not all of the steps and modules in the flows and structural diagrams above are necessary; certain steps or modules may be omitted according to actual requirements. The sequence in which the steps are executed is not fixed but may be adjusted as needed. The partitioning of the modules is merely functional partitioning, employed for the purpose of facilitating description; during actual implementation, one module may be realized by multiple modules, and the functions of multiple modules may be realized by the same module; these modules may be located in the same device, or in different devices.

Hardware modules in the aspects may be realized mechanically or electronically. For example, one hardware module may comprise a specially designed permanent circuit or logic device (such as a dedicated processor, such as an FPGA or ASIC) for completing a specific operation. The hardware module may also comprise a programmable logic device or circuit that is temporarily configured by software (e.g., comprising a general processor or another programmable processor) for executing a specific operation. The choice of whether to specifically use a mechanical method, a dedicated permanent circuit, or a temporarily configured circuit (e.g., configured by software) to realize the hardware module can be decided according to considerations of cost and time.

The present disclosure also provides a machine-readable storage medium, in which is stored an instruction for causing a machine to execute the method described herein. Specifically, a system or apparatus equipped with a storage medium may be provided; software program code realizing the function of any one of the aspects above is stored on the storage medium, and a computer (or CPU or MPU) of the system or apparatus is caused to read and execute the program code stored in the storage medium. Furthermore, it is also possible to cause an operating system, etc., operating on a computer to complete a portion of, or all, actual operations by means of an instruction based on program code. It is also possible for program code read out from the storage medium to be written into a memory installed in an expansion board inserted in the computer, or written into a memory installed in an expansion unit connected to the computer, and thereafter instructions based on the program code cause a CPU etc. installed on the expansion board or expansion unit to execute a portion of and all actual operations, so as to realize the function of any one of the aspects above. Aspects of storage media used for providing program code include floppy disks, hard disks, magneto-optical disks, optical disks (such as CD-ROM, CD-R, CD-RW, DVD-ROM, DVD-RAM, DVD-RW, DVD+RW), magnetic tapes, non-volatile memory cards and ROM. Optionally, program code may be downloaded from a server computer or a cloud via a communication network.

The aspects above are merely preferred aspects of the present disclosure, which are not intended to define the scope of protection of the present disclosure. Any amendments, equivalent substitutions or improvements etc. made within the spirit and principles of the present disclosure shall be included in the scope of protection thereof.

| Label | Meaning |
|---|---|
| 100 | Method for determining |
| | exposure parameters in X-ray |
| | imaging |
| 101-104 | Steps |
| 20 | Photography processing |
| 21 | 3D model |
| 22 | Body shape feature |
| 60 | Overall body shape feature |
| 70 | Local body shape feature |
| 221 | Upper/lower limb length ratio |
| 222 | Chest circumference |
| 223 | Waist circumference |
| 321 | Abdomen fat thickness |
| 322 | Infrasternal angle |
| 323 | Position of upper boundary of |
| | abdomen |
| 30 | State of readiness |
| 31 | 3D image |
| 32 | Body shape feature |
| 33 | Log |
| 34 | Exposure parameter |
| 35 | Exposure button depression |
| | event |
| 40 | Body shape feature |
| 41 | Exposure parameter |
| 42 | Exposure parameter prediction |
| | model |
| 43 | 3D image |
| 44 | Body shape feature determining |
| | model |
| 45 | Exposure parameter predicted |
| | value |
| 46 | Judgment process |
| 47 | Exposure parameter manual |
| | adjustment processing |
| 48 | Exposure processing |
| 500 | Apparatus for determining |
| | exposure parameters in X-ray |
| | imaging |
| 501 | Acquisition module |
| 502 | Extraction module |
| 503 | Input module |
| 504 | Receiving module |
| 505 | Exposure module |
| 506 | Update module |
| 600 | Apparatus for determining |
| | exposure parameters in X-ray |
| | imaging |
| 601 | Processor |
| 602 | Memory |

1-14. (canceled)

15. A method for determining exposure parameters in X-ray imaging, comprising:
acquiring a three-dimensional (3D) image of a subject;
based on the 3D image, extracting a body shape feature of the subject;
inputting the body shape feature into an exposure parameter prediction model corresponding to an X-ray imaging protocol, wherein the exposure parameter prediction model is obtained by training based on first training data, the first training data including a historical body shape feature in a historical exposure operation of the X-ray imaging protocol and an exposure parameter historical value in the historical exposure operation; and
receiving, from the exposure parameter prediction model, an exposure parameter predicted value determined based on the body shape feature.

16. The method as claimed in claim 15, further comprising:
establishing a first artificial neural network model;
using the first training data to train the first artificial neural network model into the exposure parameter prediction model, wherein the training comprises:
inputting the first training data into the first artificial neural network model, so that the first artificial neural network model outputs an exposure parameter predicted value corresponding to the first training data;
based on a difference between the exposure parameter historical value in the first training data and the exposure parameter predicted value corresponding to the first training data, determining a loss function value of the first artificial neural network model; and
configuring a model parameter of the first artificial neural network model, so that the loss function value is lower than a first preset threshold; and determining the first artificial neural network model resulting from the configuration to be the exposure parameter prediction model.

17. The method as claimed in claim 15, wherein the step of extracting a body shape feature of the subject based on the 3D image comprises:
inputting the 3D image into a body shape feature determining model, wherein the body shape feature determining model is obtained by training based on second training data, the second training data including a historical 3D image and a body shape feature labeled on the basis of the historical 3D image; and
receiving, from the body shape feature determining model, a body shape feature determined on the basis of the 3D image.

18. The method as claimed in claim 17, further comprising:
establishing a second artificial neural network model;
using the second training data to train the second artificial neural network model into the body shape feature determining model, the training comprising:
inputting the second training data into the second artificial neural network model, so that the second artificial neural network model outputs a predicted body shape feature corresponding to the second training data;
based on a difference between the labeled body shape feature in the second training data and the predicted body shape feature, determining a loss function value of the second artificial neural network model;
configuring a model parameter of the second artificial neural network model, so that the loss function value is lower than a second preset threshold; and
determining the second artificial neural network model resulting from the configuration to be the body shape feature determining model.

19. The method as claimed in claim 15, further comprising:
judging whether an adjustment instruction for the exposure parameter predicted value is received;
wherein, when an adjustment instruction for the exposure parameter predicted value is received, the exposure parameter predicted value is adjusted on the basis of the adjustment instruction, and the imaging target is subjected to a first exposure operation on the basis of the adjusted exposure parameter predicted value; and
when no adjustment instruction for the exposure parameter predicted value is received, the imaging target is subjected to a second exposure operation on the basis of the exposure parameter predicted value.

20. The method as claimed in claim 19, further comprising:
after executing the first exposure operation, storing third training data in a log, the third training data including the adjusted exposure parameter predicted value for executing the first exposure operation and a body shape feature of the imaging target;
extracting the third training data from the log;
using the third training data to subject the exposure parameter prediction model to an update training process, the update training process comprising:
inputting the third training data into the exposure parameter prediction model, so that the exposure parameter prediction model outputs an exposure parameter predicted value corresponding to the third training data;
based on a difference between the adjusted exposure parameter predicted value in the third training data and the exposure parameter predicted value corresponding to the third training data, determining a loss function value of the exposure parameter prediction model;
configuring a model parameter of the exposure parameter prediction model, so that the loss function value is lower than a third preset threshold; and
determining the exposure parameter prediction model resulting from the configuration to be an exposure parameter prediction model resulting from update training.

21. The method as claimed in claim 15, wherein the body shape feature comprises a first body shape feature having a first weighting and a second body shape feature having a second weighting, wherein the first body shape feature characterizes an overall body shape of the subject, and the second body shape feature characterizes a local body shape of an imaging target, which is included in the subject and corresponds to the X-ray imaging protocol, wherein the second weighting is greater than the first weighting.

22. An apparatus for determining exposure parameters in X-ray imaging, wherein the apparatus comprises:
an acquisition module, configured to acquire a 3D image of an imaging target;
an extraction module, configured to extract a body shape feature of the imaging target based on the 3D image;
an input module, configured to input the body shape feature into an exposure parameter prediction model corresponding to an X-ray imaging protocol, wherein the exposure parameter prediction model is obtained by training based on first training data, the first training data including a historical body shape feature in a historical exposure operation of the X-ray imaging protocol and an exposure parameter historical value in the historical exposure operation; and
a receiving module, configured to receive, from the exposure parameter prediction model, an exposure parameter predicted value determined on the basis of the body shape feature.

23. The apparatus as claimed in claim 22, wherein the extraction module is configured to input the 3D image into a body shape feature determining model, wherein the body shape feature determining model is obtained by training based on second training data, the second training data including a historical 3D image and a body shape feature labeled on the basis of the historical 3D image; and to receive, from the body shape feature determining model, a body shape feature determined on the basis of the 3D image.

24. The apparatus as claimed in claim 22, further comprising:
an exposure module, configured to judge whether an adjustment instruction for the exposure parameter predicted value is received, wherein, when an adjustment instruction for the exposure parameter predicted value is received, the exposure parameter predicted value is adjusted on the basis of the adjustment instruction, and the imaging target is subjected to a first exposure operation on the basis of the adjusted exposure parameter predicted value; and
when no adjustment instruction for the exposure parameter predicted value is received, the imaging target is subjected to a second exposure operation on the basis of the exposure parameter predicted value.

25. The apparatus as claimed in claim 24, further comprising:
an update module, configured to store third training data in a log after the first exposure operation is executed, the third training data including the adjusted exposure parameter predicted value for executing the first exposure operation and a body shape feature of the imaging target;
extracting the third training data from the log; using the third training data to subject the exposure parameter prediction model to an update training process, the update training process comprising: inputting the third training data into the exposure parameter prediction model, so that the exposure parameter prediction model outputs an exposure parameter predicted value corresponding to the third training data;
based on a difference between the adjusted exposure parameter predicted value in the third training data and the exposure parameter predicted value corresponding to the third training data, determining a loss function value of the exposure parameter prediction model;
configuring a model parameter of the exposure parameter prediction model, so that the loss function value is lower than a third preset threshold; and
determining the exposure parameter prediction model resulting from the configuration to be an exposure parameter prediction model resulting from update training.

26. An apparatus for determining exposure parameters in X-ray imaging, comprising:
a processor; and
a memory,
wherein an application program executable by the processor is stored in the memory, and used to cause the processor to perform the method for determining exposure parameters in X-ray imaging as claimed in claim 15.

27. A non-transitory computer-readable storage medium, having stored therein a computer-readable instruction which, when executed by a processor, realizes the method for determining exposure parameters in X-ray imaging as claimed in claim 15.
